(19) United States
(12) Patent Application Publication
(10) Pub. No.: US 2006/0235528 A1
(43) Pub. Date: Oct. 19, 2006

(54) Angled sliding core, also as part of an intervertebral disc prosthesis, for the lumbar and cervical spine

(76) Inventor: Karin Buettner-Janz, Berlin (DE)

Correspondence Address: JOYCE VON NATZMER;Hall, Vande Sande & Pequigot, LLP, 10220 River Road, Suite 200, Potomac, MD, 20854, US

(21) Appl. No.: 11/379,099

(22) Filed: Apr. 18, 2006

(30) Foreign Application Priority Data
Oct. 18, 2005 (WO) PCT/DE05/01883
Oct. 18, 2004 (WO) PCT/DE04/02330

(51) Int. Cl. A61F 2/44

(52) U.S. Cl. 623/17.14; 623/17.15

(57) ABSTRACT

The invention relates to a sliding core and an intervertebral disc prosthesis for the compensation of angles between vertebral endplates, for the preservation or improvement of function of a motion segment of the lumbar or cervical spine. A sliding core, according to the invention, for functional two- or three part intervertebral disc prostheses, is intended to ascertain a compensation, for the correction or preservation of angles within an intervertebral space. By this, it is possible not to have to remove implanted prosthetic plates from their assembly to vertebral bodies. According to the invention, functional two- and three part intervertebral disc prostheses with an asymmetrically angled sliding core are also planned. Concomitantly, upper and lower sliding partner of a three component prosthesis as well as the two sliding partners of a two part prosthesis function as endplates, which have means for a firm assembly to an upper and lower vertebral body.

### CROSS REFERENCE SECTION

This is a continuation-in-part application of international application no. PCT/DE2005/001883, filed Oct. 18, 2005 designating the U.S. and claiming priority from international application no. PCT/DE2004/002330, filed Oct. 18, 2004. Both of these applications are incorporated herein by reference in their entirety.

### BACKGROUND OF THE INVENTION

The invention relates to an intervertebral disc prosthesis for the total replacement of an intervertebral disc of the lumbar and cervical spine.

The idea of function-retaining artificial replacements for intervertebral discs is younger than that for replacements of artificial joints of extremities, but in the meantime about 50 years old [Büttner-Janz, Hochschuler, McAfee (Eds.): The Artificial Disc. Springer Verlag, Berlin, Heidelberg, New York 2003]. It results from biomechanical considerations, unsatisfactory results of fusion surgeries, disorders adjacent to fusion segments and from the development of new materials with greater longevity.

The publications and other materials, including patents, used herein to illustrate the invention and, in particular, to provide additional details respecting the practice are incorporated herein by reference.

By means of function-retaining disc implants it is possible to avoid fusion surgery, i.e. to maintain, or to restore the mobility within the intervertebral disc space. In an in-vitro experiment it is also possible to achieve a normalization of the biomechanical properties of the motion segment to a large extent through the implantation of an artificial intervertebral disc after a nucleotomy.

Implants for the replacement of the whole intervertebral disc differ from those for the replacement of the nucleus pulposus. Accordingly, implants for the total replacement of the intervertebral disc are voluminous; they are implanted via a ventral approach. An implantation of a prosthesis for total replacement of the intervertebral disc immediately after a standard nucleotomy can therefore not be carried out.

The indication for a function-retaining intervertebral disc replacement as an alternative to the surgical fusion includes, besides the painful discopathy, also pre-operated patients with a so-called post discectomy syndrome, patients with a recurrent herniated intervertebral disc within the same segment and patients having a pathology within the neighbouring intervertebral disc as a consequence fusion surgery.

Presently, a total of more than 10 different prostheses are clinically used for the total replacement of intervertebral discs. For the lumbar spine the Charité Artificial Disc, the Prodisc, the Maverick, the FlexiCore and the Mobidisc (Overview in Clinica Reports, PJB Publications Ltd., June 2004) are particularly well known, and for the cervical spine the Bryan prosthesis, the Prestige LP prosthesis, the Prodisc-C and the PCM prosthesis, which will be described below.

The Prodisc prosthesis for the lumbar spine is being implanted since 1999, following its further development to the Prodisc II. Although with respect to its components a three-part intervertebral disc prosthesis, it is functionally a two-part prosthesis with its sliding partners made of metal and polyethylene. Implantations of the Prodisc are carried out in the lumbar spine and with an adapted model of the prosthesis, the Prodisc-C, also in the cervical spine. Different sizes, heights (achieved by the polyethylene core) and angles of lordosis (achieved by the metal endplates) are available. Bending forward and backward as well as to the right and left is possible to the same extent of motion; the axial rotation is not limited in the construction.

The same applies to both two-part prostheses for the cervical spine, the PCM prosthesis with its sliding partners metal and polyethylene and the Prestige LP prosthesis with its sliding partners metal-metal. As special feature of the construction of the Prestige LP prosthesis it has the possibility for an anterior-posterior translation, due to the horizontal ventrally prolonged concavity, which, in a frontal section, has the same radius as the convexity.

The Maverick and the FlexiCore for the lumbar spine are functionally two-part prostheses with spherical convex-concave sliding partners, both with sliding partners made of metal-metal. In contrast, the Mobidisc is functionally a three-part prosthesis with sliding partners of metal-polyethylene and two articulation surfaces. One area is a segment of a sphere, as it is in the three afore mentioned prostheses, with a convex and a concave surface of the articulating partners each of the same radius, the other area of the Mobidisc being plane. Although a limitation of the axial rotation is planned within the plane section, it is not limited within the convex-concave area of articulation. In contrast the FlexiCore has a small stopping area within the spherical sliding surfaces limiting the rotation movement.

The Bryan prosthesis is clinically used as a compact prosthesis for total replacement of intervertebral discs of the cervical spine. It is attached to the vertebral bodies by convex titanium plates with a porous surface and achieves its biomechanical properties by virtue of a polyurethane nucleus.

The longest experience exists with the Charité prosthesis, which is matter of the DE 35 29 761 C2 and the U.S. Pat. No. 5,401,269. This prosthesis was developed in 1982 by Dr. Schellnack und Dr. Büttner-Janz at the Charité in Berlin and was later on named SB Charité prosthesis. In 1984 the first surgery took place. The intervertebral disc prosthesis was further developed and since 1987 the current type of this prosthesis, model III, is being implanted; in the meantime over 10000 times worldwide (DE 35 29 761 C2, U.S. Pat. No. 5,401,269). The prosthesis is functionally three-parted with the sliding partners metal and polyethylene with two identical spherical sliding surfaces. On the one hand it has a transversally mobile polyethylene core and on the other hand the accordingly adapted concave cups within the two metal endplates. For the adaptation to the intervertebral space, the Charité prosthesis provides different sizes of the metal plates and different heights of the size adapted sliding cores as well as angled prosthetic endplates, which when implanted vice versa in sagittal direction can also be used as replacement for the vertebral body. The primary fixation of the Charité prosthesis is achieved by six teeth, which are located in groups of three slightly towards the middle next to the frontal and rear edge of each prosthetic plate.

The other prostheses have other primary fixations on their towards the intervertebral bodies directed surfaces, e.g. a sagittally running keel, a structured surface, a convex shape with for instance crosswise running grooves and combinations thereof, also with differently located teeth. Furthermore screw fixations can be used, either from ventral or from within the intervertebral space into the intervertebral body.

To assure a long-term fixation of the prosthetic endplates to the intervertebral bodies and to thus generate a firm connection with the bone, a surface was created in analogy to cement-free hip and knee prostheses, which combines chrome-cobalt, titanium and calcium phosphate in such a way that it is possible for bone to grow directly onto the endplates. This direct connection between prosthesis and bone, without the development of connective tissue, makes a long-term fixation of the artificial intervertebral disc possible and reduces the danger of loosening or displacements of the prosthesis and material breakage.

One primary objective of function retaining intervertebral disc replacements is to closely adapt the motions of the prosthesis to the ones of a healthy intervertebral disc. Directly connected to this is the motion and stress for the facet joints, which following inappropriate biomechanical stress have their own potential for disorders. There can be abrasion of the facet joints (arthritis, spondylarthritis), in the full blown picture, with the formation of osteophytes. As result of these osteophytes and also by a pathologic course of motion of the intervertebral disc alone, the irritation of neural structures is possible.

The healthy intervertebral disc is, in its interactions with other elements of the motion segment, composed in such a way that it allows only motions to a certain extent. For example, within the intervertebral disc, motions to the front and back are combined with rotary motions, and side motions are also combined with other motions. The motion amplitudes of a healthy intervertebral disc are very different, with respect to the extension (reclination) and flexion (bending forward) as well as to the lateral bending (right and left) and rotary motion. Although of common basic characteristics, there are differences between the motion amplitudes of the lumbar and cervical spine.

During motion of the intervertebral disc the centre of rotation changes, i.e. the motion of the intervertebral disc does not take place around a fixed center. Due to a simultaneous translation movement of the adjacent vertebrae, the center changes its position constantly (inconstant center of rotation). The prosthesis according to DE 35 29 761 C2 shows a construction which differs in comparison to other available types of prostheses which are build like a ball and socket joint, as a result of which they move around a defined localized centre of rotation. By virtue of the three-part assembly of the prosthesis according to DE 35 29 761 C2, with two metallic endplates and the interpositioned freely mobile polyethylene sliding core, the course of motion of a healthy intervertebral disc of the human spine is mimicked as far as possible, however without the exact motion amplitudes in the specific motion directions.

A further important feature of the healthy lumbar intervertebral disc is its trapezium shape, which is primarily responsible for the lordosis of the lumbar and cervical spine. The vertebral bodies themselves contribute only to a minor extent to the lordosis. During prosthetic replacement of intervertebral discs the lordosis should be maintained or reconstructed. The Charité disc prosthesis provides four differently angled endplates, which moreover can be combined with each other. However during surgery there is more surgical effort and the risk to damage the vertebral endplates with the resulting danger of subsidence of the prosthesis into the vertebral bodies, if the prosthesis has to be removed completely, because a good adjustment of lordosis and an optimal load of the center of the polyethylene core were not achieved.

To avoid sliding or a slip-out of the middle sliding partner from the endplates, the DE 35 29 761 C2 discloses a sliding core with a two-sided partly spherical surface (lenticular), with a plane leading edge and at the exterior with a ring bulge, which will lock between the form-adapted endplates during extreme motion. The DE 102 42 329 A1 discloses a similar intervertebral disc prosthesis which has a groove around the contact surfaces, in which an elastic ring is embedded that is in contact with the opposite contact area for a better course.

The EP 0 560 141 B1 describes a three-part intervertebral disc prosthesis, which also comprises two endplates and an interpositioned prosthetic core. The intervertebral disc prosthesis, described in this document, provides a resistance during rotation of its endplates in opposing directions around a vertical rotary axis without a contact between the prosthetic endplates. This is achieved by a soft limitation of the endplates during rotation onto the prosthesis core caused by the weight, which acts on the plates as a result of the biomechanical load transfer within the spine, because the corresponding radii of curvature differ in a median-sagittal and frontal transection.

The above mentioned models are permanently anchored in the intervertebral spaces as implants. Especially due to a load transfer over too small surface areas, a migration of the endplates into the vertebral bodies and thus a dislocation of the complete implant is possible in middle to long-term, resulting in artificial stress for the vertebral bodies and the adjacent nerves and in the end for the total motion segment, and leading to new complaints of the patients. The long-term stability of the polyethylene and the restricted mobility of the intervertebral disc prosthesis due to an inappropriate load on the polyethylene within the intervertebral space have to be discussed. Insufficiently adapted ranges of motion and adverse biomechanical stress in the motion segment can possibly lead to persistence of the complaints or later on to new complaints of the patients.

The U.S. Pat. No. 6,706,068 B2 on the other hand, describes an intervertebral disc prosthesis comprising an upper and lower part, in which the parts are built correspondently towards each other. No intermediate part as middle sliding partner exists. Different designs are realized for the interdigitating and articulating partners, resulting in a two-part prosthesis. The design is however limited to structures having either edges or corners so that this way both parts of the prosthesis articulate with each other; in this case it is not possible to speak of sliding partners. Furthermore two sliding partners are described having one convex part towards the interior of the prosthesis and the other sliding partner is correspondingly shaped concavely. This kind of prosthesis, however, allows restricted movements of the artificial intervertebral disc only. The concave protuberance corresponds to a part of a ball with the according radius. The U.S. Pat. No. 6,706,068 B2 further shows a two-part disc prosthesis having convex and concave partial areas on each sliding partner corresponding to concave and convex partial areas of the other sliding partner.

According to the disclosure of the U.S. Pat. No. 6,706,068 B2 several fixed points of rotation are generated.

The US 2004/093085 describes an implant for the intervertebral space, which has asymmetrical ends, which is adapted to the bow-shaped peripheral circumference of a natural intervertebral disc, and is visible in the transverse cross section. By virtue of this it is meant to assure that such an implant can cover a maximal area between the neighbouring vertebrae, without jutting out beyond the outside edges. The ends of an implant, according to US 2004/093085 may also be flattened, so that they can reach into the periphery of the intervertebral space and be more easily introduced into the intervertebral space.

From the present state of the arts asymmetrical, in ventrodorsal direction angular prosthetic plates are known (e.g. Charité Artificial Disc, Mobidisc), which are meant to compensate inclinations of adjacent vertebral bodies towards each other. Oblique prosthetic plates lead to a better adaptation to the anatomic and biomechanic conditions of the motion segment, particularly in the lumbar spine, which has the greatest number of disorders. It is above all there that considerable differences between the ventrodorsal angles between individual intervertebral discs exist. The implantation of such prostheses can, however, lead to an uneven load distribution on the sliding surfaces. This results in a higher level of wear of the materials and to a reduced mobility of the prosthesis as well as to disadvantages for the facet joints (see above). Furthermore an exchange of the endplates, for instance because the inclination of the prosthetic plates are not exact, is mostly associated with damage to the bone of the respective vertebra together with a higher risk of causing damage to the large blood vessels. Added to that the assortment of angled prosthetic plates is usually not sufficient to assure an optimal implantation of the prosthesis, and in the case of a revision of the prosthetic plates, the resulting soft-tissue tonus of the intervertebral space again leads to no optimal angles, due to the manipulation during the explantation.

Thus, there is a need for a sliding core or an intervertebral disc prosthesis for the total replacement of intervertebral discs, which is suited to compensate the angles of inclination between the vertebral endplates for the purpose of maintaining or improving the function of a motion segment of the lumbar and cervical spine.

As per invention this need is addressed by an asymmetrically angled sliding core and a prosthesis with an asymmetrically angled sliding core are intended.

### SUMMARY OF THE INVENTION

With respect to the present invention the three body axes are described by the following terms: A “sagittal section” or a view in the “sagittal plane” allows a lateral view, because the section plane runs vertically from the front to the back. The term “front” is synonymous “ventral” and the term “back” to “dorsal”, because using these terms, the orientation of the prosthesis within the body is indicated. A “frontal section” or the “frontal plane” is a vertical cross-section from one side to the other. The term “lateral” stands for sidewise. Sagittal and frontal sections are vertical sections as they both run in a vertical plane, but 90 degree displaced from one another. A view in the “transversal plane” or a “transversal section” shows a top-view onto the prosthesis, because it is a horizontal section.

Concomitant with the description and depiction of the present invention an articulation area signifies that region of the sliding partners, which comprises the curved convex, concave and plane parts of the surfaces, which come into contact or articulate with each other. Because of this the articulation area is synonymous with the term sliding area.

The term “corresponding”, with respect to the articulating sliding surfaces designates not only congruent convex and concave shaped surfaces articulating with each other. Moreover this term also designates articulating surfaces that are not completely congruent. Such “deviations” or tolerances regarding the sliding surfaces of articulating sliding partners can be caused on the one hand by the chosen materials and shapes. On the other hand it may also be intended that convexity and the concavity articulating with it are not totally congruent, for instance in order to designate the respectively wished for possibilities of motion of the articulating partners directly.

For this invention, the term sliding core and middle sliding partner are to be understood synonymously with respect to a three part intervertebral disc prosthesis. The invention expressly also refers to sliding cores, which as result of their assembly, without sliding area, to an upper or lower sliding partner, are factually part of a two part prosthesis and whose opposite side articulates with the second sliding partner.

As per invention, a sliding core, which is positioned between upper and lower sliding partner of an intervertebral disc prosthesis for the compensation of angles of inclination between vertebral endplates to maintain or improve the function of a motion segment of the lumbar and cervical spine, is intended and characterized by the fact that, depending on the design of a convexity and/or concavity on the upper and/or lower side of the sliding core, one or two articulating sliding surface(s) are formed between the sliding core and the inside(s) of the upper and/or lower sliding partner and that the sliding core is designed asymmetrically in such a way that at least one sliding surface of the sliding core is inclined in a definite angle towards a fictitious horizontal in at least one vertical cross-section.

The inclination towards a horizontal of at least one of the sliding surfaces of the sliding core will be also described as inclination of the sliding core in the following. As per invention, a sliding core is thus not only designed asymmetrically, but also purposely inclined.

As per invention, a sliding core is intended for functional two- and three part intervertebral disc prosthesis, to compensate, to correct, or maintain angular asymmetries within an intervertebral space. This also makes it possible to perform an exact angle reconstruction of the intervertebral space, so that implanted, where applicable, angled prosthetic plates do not need to be removed from their anchoring with the vertebral bodies again. This not only leads to better treatment results, but also to much shorter surgery times. In the case the sliding surfaces of the sliding core correspond with the sliding partners that are assembled to the vertebral bodies, a sliding core, as per invention, can be purposely selected and implanted from an assortment of differing surface areas as well as different heights and different angles of sliding cores with respect to its asymmetrical design. As per invention, a sliding core can thus be designed in such way, that it can articulate with the sliding partners of already present intervertebral disc prostheses, its use being indicated or advantageous because of its asymmetry.

The angles of the intervertebral space between two adjacent vertebral endplates lie between minus 10 degrees, including about minus 5, about 0, about plus 5, about plus, 10, about plus 15, about plus, 20, about plus 25, about plus 30 degrees and plus 35 degrees, with negative degrees indicating a pathological kyphosis of the intervertebral space, the opposite of a physiological lordosis. In the case of a lordosis, the higher side of the sliding core points ventrally and dorsally in the case of kyphosis. It is the surgical objective, to intra-operatively produce a position of the sliding partners using the sliding core, as per invention, which shows only a minimal or no inclination of the sliding partners towards each other as a prerequisite for a post-operative mobility of the intervertebral space, which is near to physiological conditions, so that the facet joints can be protected and the neighboring intervertebral discs relieved. In case of no previous inclination of the sliding partners, the motion amplitudes in the different directions are postoperatively optimally possible. As the main objective of function retaining intervertebral disc prosthesis is to maintain the mobility of the intervertebral space, the sliding core, as per invention, thus plays a key role.

As per invention, the sliding core refers to an angular range of between 2 degrees and 35 degrees, with intended steps of 2 degrees to 5 degrees for the sliding core. The angular range may be between about 4 degrees, about 6 degrees, about 8 degrees, about 10 degrees, about 12 degrees, about 14 degrees, about 16 degrees, about 18 degrees, about 20 degrees, about 22 degrees, about 24 degrees, about 26 degrees, about 28 degrees, about 30 degrees and about 32 degrees. After the angle of the intervertebral space has been intra-operatively quantified with an adapted trial sliding core or a suited instrument the most suitable sliding core is implanted and its alignment adapted to the position of the corresponding sliding partner(s). In the case of a kyphosis of the intervertebral space as a starting point, it can be intra-operatively decided, whether in the best case a lordosis can be created through the implantation of a sliding core, as per invention, or whether to try to at least reduce the kyphosis using a sliding core with a small angle, with the implanted core having only a small, dorsally open angle.

In a preferred design of the sliding core, as per invention, the convexity and/or concavity spans across the whole upper and/or lower side of the sliding core or is surrounded in each case by a edge, whose breadth and height are equal or different. The sliding core, as per invention, is thus intended with a edge as well as without one.

A edge, in the sense of the invention, indicates an area located between outer edge of a sliding core or sliding partner and convexity(ies) or concavity(ies) belonging to it. The edges of the respective sliding partners run horizontally and/or obliquely and preferably have a plane surface. It is essential for the design of the surfaces of the edges, that during terminal inclination of the sliding partners towards each other a maximally possible contact between the edges of the sliding partners is guaranteed. Should the edges not have a plane surface, they have to in any case be designed in such a way that when they close towards each other, a maximally possible contact arises between them.

In the case of a one-sided design of a sliding area, suitable means for a permanent, or a permanent but reversible assembly with an upper or lower sliding partner are intended for the opposite side. It is however also intended that an asymmetrically angled sliding core with a one-sided sliding surface has means for a permanent or permanent but reversible assembly with a further symmetrically or asymmetrically angled sliding core with a one-sided sliding surface. This assembly results in a sliding core with sliding surfaces on the upper and lower side, which is suited for a functional three part intervertebral disc prosthesis.

The means for an assembly with a sliding partner or between sliding cores with one sided sliding surfaces, are presented in particular by a thinning or flat broadenings, perhaps also including the edges. Generally speaking, the shape of a sliding core, as per invention, is also adapted to the respective means for assembly. For such an assembly between the sliding core and the upper or lower sliding partner, a tongue and groove assembly, a guiding track and corresponding recess, a snap mechanism, gluing and screwing are intended.

For a sliding core, as per invention, it is intended that the whole sliding core or the articulating sliding surface(s)—in as much as the sliding surface(s) do not extend up to the outer periphery(ies)—the edge and, where applicable, the means for assembly of the sliding partners—are each made of the same or different materials as the articulating sliding partners, or are equally or differently coated as these.

It is further intended that a sliding core, as per invention, has as a stop on its outside to prevent a slip out from within an intervertebral disc prosthesis during terminal gap closure of the sliding partners. This stop is higher than the sliding core or its edge at least on the upper or lower side of the sliding core.

The stop of a sliding core against a slip out from within the intervertebral disc prosthesis during terminal gap-closure of the sliding partners which is on the upper or lower side of the sliding core may, as per invention, be designed in such a way that it is equal to or higher than the sliding core or its edge and is lead within a tongue of the peripheral region of the upper and/or lower sliding partner with the necessary liberty for the maximal sliding motion of the sliding partner.

In a further version of the sliding core with a edge, as per invention, it is intended that the height of edge partly of completely continuously increases beginning from the transition area between the convexity and the edge up until the peripheral edge area. This is intended without the size of the aperture angle changing as a result of an adaptation to the height of the edge of an upper and lower sliding partner of a three part intervertebral disc prosthesis. This “dovetail” shape of the edge of the sliding core increases the safety against dislocation.

As per invention, a sliding core has a sliding surface made of plane, spherical, cylindrical, ellipsoid, spindle-shaped, oval or asymmetrical surfaces or combinations thereof, which are suited for a sliding motion, with the sliding core with sliding surfaces on the upper and lower side having identical or non-identical sliding surfaces with respect to shape, height and/or direction of the possible sliding motions. In this invention, “spindle-shaped” refers to a shape that is similar to that of an American football. By virtue of the flexible shaping of the articulating surfaces of the sliding core, as per invention, its adaptation to the design of the concavity(ies) and or convexity(ies) of existing sliding partners, which are assembled to a vertebral body, is made possible. An asymmetrically designed, angled sliding core can thus also be fitted to the articulation surfaces of existing prostheses. This opens up the possibility, to make sliding cores for different types of prostheses available and to take into account existing asymmetries of the respective intervertebral space by a design of non-parallel sliding surfaces or to purposefully “set” the motion in such a way as to protect the border between the vertebral body and the implant and especially the facet joints.

As the angle of a sliding core with 2 articulating surfaces can be the same or different above and below, with respect to a fictitious horizontal, a maximal flexibility regarding the adaptation of a sliding core, as per invention, to the respective intervertebral space is possible.

A further matter of the invention is an intervertebral disc prosthesis for the compensation of angles between vertebral endplates to maintain or restore the function of a motion segment of the lumbar and cervical spine, comprising an upper sliding partner with an upper outer side, which has means for an assembly with an upper vertebral body and a lower sliding partner with a lower outer side, which has means for an assembly with a lower vertebral body, where between the inner sides of the upper and lower sliding partner a sliding core is positioned, which is characterised by the fact that depending on the design of a convexity and/or concavity on the upper and/or lower side, one or two articulating surfaces arise between the sliding core and the inner side(s) of the upper and/or lower sliding partner and the sliding core is designed asymmetrically in such a way that in at least one vertical cross-section at least one sliding surface of the sliding core is inclined in a definite angle towards a fictitious horizontal.

As per invention, a functional two- or three part intervertebral disc prosthesis with an asymmetrically angled sliding core is intended. Upper and lower sliding partner of a three part prosthesis as well as the two sliding partners of a two part prosthesis at the same function as endplates, which have means for an assembly with an upper or lower vertebral body.

An important advantage of the functional two- and three part intervertebral disc prosthesis is the creation of a possibility to correct or to maintain asymmetries of angles of an intervertebral space, without the need to remove previously implanted prosthetic plates from their fixation to vertebral bodies again, provided correspondingly formed convexities and/or concavities of sliding core and sliding partner(s) as well as suitable edge design and means for an assembly are present, where applicable.

The angles of the intervertebral space between two adjacent vertebral endplates lie between minus 10 degrees, including about minus 5, about 0, about plus 5, about plus, 10, about plus 15, about plus, 20, about plus 25, about plus 30 degrees and plus 35 degrees, with negative degrees indicating a pathological kyphosis of the intervertebral space, the opposite of a physiological lordosis. In the case of a lordosis the higher side of the sliding core is points ventrally and dorsally in the case of kyphosis. It is the surgical objective, to intra-operatively produce a position of the sliding partners using the sliding core, as per invention. This position shows only a minimal or no inclination of the sliding partners towards each other as a prerequisite for a post-operative mobility of the intervertebral space, which is near to physiological conditions, so that the facet joints can be protected and the neighboring intervertebral discs relieved. In case of no previous inclination of the sliding partners, the motion amplitude in the different directions is postoperatively optimally possible.

The sliding core within the intervertebral disc prosthesis on the whole refers to an angular range of between 2 degrees and 35 degrees, with intended steps of inclinations of 2 degrees to 5 degrees for the sliding core. The angular range may be between about 4 degrees, about 6 degrees, about 8 degrees, about 10 degrees, about 12 degrees, about 14 degrees, about 16 degrees, about 18 degrees, about 20 degrees, about 22 degrees, about 24 degrees, about 26 degrees, about 28 degrees, about 30 degrees and about 32 degrees. After the angle of the intervertebral has been intra-operatively quantified with an adapted trial sliding core or another suited instrument the most suitable sliding core is implanted and its alignment adapted to the position of the corresponding sliding partner(s). In the case of a kyphosis of the intervertebral space as a starting point, it can be intra-operatively decided, whether in the best case a lordosis can be created through the implantation of a sliding core, as per invention, or whether to try to at least reduce the kyphosis using a sliding core with a small angle, with the implanted core having only a small, dorsally open angle.

It is, for instance, thus possible to select and implant a sliding core with a suitable asymmetry still during the operation, in order to optimally adapt to or correct the preoperatively existing angle. Further the possibility arises to compensate changes to pathologic positions, which have arisen either during the operation or in the course of the years, by using a another sliding core. As in the case of a functional three part prosthesis or functional two-part prosthesis, with a sliding core that can be implanted separately onto the upper or lower prosthetic plate so that both components have a permanent or permanent, but reversible assembly, the prosthetic plates do not need to be exchanged, there is also no need to fear damaging of the vertebral bodies. Added to that, well “ingrown” prosthetic plates will not have to be removed from their connection with the respective vertebral body during second surgery, so that again no damage to the vertebral body with a postoperatively greater risk of subsidence of the prosthesis into the vertebral body and thus no therapeutic failure, takes place. Beyond that the assortment of prosthetic plates can be kept smaller, because angular prosthetic plates will be replaced by asymmetrically angled sliding cores.

As per invention, the intervertebral disc prostheses further offer the possibility of maintaining or correcting an individual scoliosis of a patient within the surgical segment, without disadvantages to the range of motion of the prosthesis or strain on the facet joints arising. During an operation to implant an intervertebral disc prosthesis, the operation enables the scoliotic asymmetrical distraction of the intervertebral space prior to the implantation of the prosthetic plates. With an intervertebral disc prosthesis, as per invention, with an asymmetrically angled sliding core, the possibility is given to adapt the sliding surfaces to these asymmetries. This facilitates an optimal motion of the operated motion segment for the patient because the corresponding convex and concave sliding surface, for instance, stand in a middle position; i.e. without any inclination of the prosthetic components towards each other as a starting point for the movements to the different directions. The patient does not need to apply any increased forces to carry out a motion from an already inclined starting point of the prosthesis into the opposing direction, which would include an intervertebral distraction for the purpose of overcoming the height resulting from the convexity.

In the case of symmetrically or insufficiently inclined sliding partners of the prosthesis, it comes to an uneven distribution of load onto the sliding partners; particularly in terminal angular positions, which results in higher one-sided forces. This again leads to increased stress of the parts of the prosthesis, exposing them to higher wear. The measure of an angled sliding core, as per invention, thus leads to a protection of the materials, including the surfaces of the parts of the prosthesis of the intervertebral disc prosthesis, as per invention.

In a two- or three part intervertebral disc prosthesis, as per invention, the articulation surfaces of the upper and lower sliding partner are each surrounded be a edge of equal or different breadth and height. In the case of a three part prosthesis, the articulation surfaces of the sliding core each span across the whole upper and lower sides, i.e. without a edge, or the articulation surfaces are each surrounded by a edge of equal or different breadth and height.

For an intervertebral disc prosthesis as per invention, a edge is of especial advantage when it is involved in gap-closure during terminal inclination because the load on the motion segment is distributed over a larger surface area. This results in further protection of the material of the parts of the prosthesis or the coating of the surfaces. Added to that, a edge, which surrounds the corresponding articulation surfaces, can also help in the building of the inclination.

It is intended for an intervertebral disc prosthesis, as per invention, that the asymmetrically angled sliding core and the sliding partners are each constructed in one piece or that the sliding partner, and/or the asymmetrically angled sliding core each comprise two permanent or permanent, but reversibly assembled parts or that the asymmetrically angled sliding core is permanently or permanently but reversibly assembled to one of the sliding partners and the opposing side of the corresponding articulation surface have means for a permanent or permanent but reversible assembly and the sliding partner and/or the sliding core as well as the parts assembled to it are made of the same or different materials and that the surfaces are either equally or differently coated.

As per invention, adaptations of the shape of assembled parts or, for instance the convexity or concavity of opposing side, such as flat broadenings, which are part of the edge or the complete edge, or recesses, are intended as suitable means for an assembly. Each sliding partner and/or for instance the convexity and/or concavity as well as the edge are intended as parts which can be assembled, depending on the design. In the case of a middle sliding partner, it is intended that this results from the assembly of the respective parts.

In the case an intervertebral disc prosthesis, as per invention, comprises permanently or permanently but reversibly assembled parts, the assembly between the sliding partner and for instance the convexity(ies) or concavity(ies) using a tongue and groove assembly, a guiding track and corresponding recesses, a snap mechanism, gluing and screwing is intended.

Regarding the materials of a two- and three part intervertebral disc prosthesis, as per invention, it is not only intended that the asymmetrically angled sliding core and the sliding partners are made of the same or different materials or that the surfaces are equally of differently coated, but also that the asymmetrically angled sliding core may comprise many or one material(s), depending on the one hand on whether different functional regions, such as edge or middle portion are built for an assembly, or on the other hand on the materials of the articulation sliding partners.

The sliding partners of an intervertebral disc prosthesis, as per invention and also of a sliding core, as per invention, are manufactured from well established materials in implantation techniques; for instance upper and lower sliding partner are made of rust free metal and the middle sliding partner of medicinal polyethylene. Other combinations of materials are also feasible. The use of other alloplastic materials, which may also be bio-active or blunt, is also feasible. The sliding partners are high gloss polished on their communicating contact areas to minimize abrasion (low-friction principle). Furthermore a coating of the particular sliding partner with appropriate materials is also planned. Favoured materials are: titanium, titanium alloys, titanium carbide, alloys of cobalt and chrome or other appropriate metals, tantalum or appropriate tantalum alloys, suitable ceramic materials as well as suitable plastics or compound materials.

In a favoured versions of an intervertebral disc prosthesis, as per invention, the sliding surfaces can be made of plane, spherical, cylindrical, ellipsoid, spindle-shaped, or oval surfaces or combinations thereof, which are suited for a sliding motion, with the sliding core with sliding surfaces on the upper and lower side having identical or non-identical sliding surfaces with respect to shape, height and/or direction of the possible sliding motions. Any shapes of the sliding surfaces with respect to the convexity and/or concavity as well as plane sliding surfaces are feasible, that can enable a sliding motion. In the case a sliding core has an articulating surface on its upper and lower sides with the insides of the prosthetic plates, these sliding surfaces are not required to have identical shapes.

On the whole the maximally possible angle of inclination (aperture angle) of an intervertebral disc prosthesis, as per invention, between upper and lower sliding partner depends
a. the design of the convexity(ies) and corresponding concavity(ies) with respect to the geometry of the sliding surface, height and radius of curvature, and
b. the shape and extent of the angled asymmetry of the sliding core, and
c. the design of the edge.

For an intervertebral disc prosthesis, as per invention, a maximal aperture angle of 6°-10° including, for example 6°-7°, 6°-8°, 6°-9°, 7°-8°, 7°-9°, 7°-10°, 8°-9° or 8°-10° during one-sided gap-closure of the sliding partners during extension or flexion, and of 3°-6° including, for example 4°-6°, 5°-6°, 3°-4°, 3°-5° or 4°-5° during one-sided lateral gap-closure is intended. These maximally possible angles of inclination of the sliding partners towards each other lie within the average range of the angles of a motion segment that can be found in a healthy spine. To compensate for the tolerances within the motion segment an additional 3° will be included for every direction of motion.

Furthermore, a shift of up to 4 mm away from a midline frontal section to dorsal of the convexity(ies) and corresponding concavity(ies) is intended in a two- and three-part intervertebral disc prostheses, as per invention. Such a dorsally displaced centre of rotation corresponds to the physiological situation of the transition between lumbar spine and sacral bone, so that an approximation of the physiological situation is achieved with the intervertebral disc prosthesis, as per invention.

It is further intended that the edges of the sliding partners are outwardly close rectangularly, otherwise inclined, curved, or combined straight, curved and/or angled. Particularly in the case of a three part prosthesis, a design is feasible, in which the upper and lower side of the sliding core simply end perpendicularly or curved towards each other in their periphery and in which the breadth of the edge is not substantially differently designed compared to the upper and lower sliding partner. Thus the sliding core can remain in between the upper and lower sliding partners during terminal inclination too. By virtue of this a compact and economic (w.r.t. space) construction of an intervertebral disc prosthesis, as per invention, is possible.

A slip out of the middle sliding partner out of this “compact” design of a three part intervertebral disc prosthesis, as per invention, is on one hand prevented by the motion adapted heights of the convexity(ies) and the corresponding concavity(ies) starting with the edge around the articulation areas and on the other hand by the gap-closure between the edges of the sliding partners at terminal inclination. The convexities are designed in such a way that they will interdigitate deeply enough into the articulating concavities. A sufficient opening of the whole prosthesis post-operatively, which is a prerequisite for a slip out of the middle sliding partner, is thus not possible. An extra version to avoid a luxation of the sliding core is a stop on the other articulating partner(s), which will stop the motion of the sliding core.

Furthermore it is intended as per invention, that in the case of a middle sliding partner of a three part prosthesis, as an additional safeguard, a stop against a slip-out, slip-away or slip-aside (luxation) out of the prosthesis during gap-closure of all three sliding partners is provided. This is part of the outer edge of the middle sliding partner or the sliding core. The stop of the middle sliding partner is located next to the periphery of the upper and/or lower sliding partner and it is higher at least on the upper or the lower side than the edge of the middle sliding partner.

This stop, as an additional safeguard against a slip-out, slip-away or slip-aside (luxation) out of the prosthesis can as per invention also be designed in such a way that it is a part of the edge of the middle sliding core. It is higher than the edge of the middle sliding partner at the upper or lower side and is lead within a groove in the edge of the upper and/or the lower sliding partner with the necessary liberty for the maximal sliding motion of the sliding partners.

As per invention, a stop is an outwardly directed extension of the edge of a middle sliding partner or sliding core, which, because of its embodiment, as result of its design, is suited to prevent a slip-out of the middle sliding partner out of the concavities of the upper and lower sliding partner. It is not necessary that the stop encloses the middle sliding partner completely, because this could result in a limitation of the maximal mobility of all sliding partners. Where required, it is arranged in definite distances or opposite of positions of the edge, which represent possible positions for a slip-out of the middle sliding partner. If the stop is higher on the upper and lower side than the edge of the middle sliding partner, it can for instance be shaped like a drawing-pin, sticking with the tip from outside into the edge, so that the head of the drawing-pin juts out over the upper and lower edge of the middle sliding partner and prevents a slip-out of the middle sliding partner during a terminal inclination in direction of the drawing pin by stopping its movement via contact to the upper and lower sliding partner.

If a stop, as a safeguard to prevent slip-out, is part of the edge of the sliding partners, the height of the convexity depends only—with regard to the anatomy and the material properties—on the desired maximal inclination angles, which is also influenced by this (see above).

A stop to secure the middle sliding core of a three-part prosthesis is advantageously shaped in such a way that it is part of the contact areas during terminal inclination of the edges of the sliding partners. Due to this fact the stop functions not only as a safeguard, but additionally it increases the load bearing area during terminal inclination of the sliding partners; the advantages of this have been described above. The possibility for such a design, however, depends crucially on the external shape and the respective breadth of the edge of the convexity and concavity of the upper and lowers sliding partners.

In a further design of a three part intervertebral disc prosthesis it is intended that the height of the middle sliding partner or sliding core partly or totally continuously increases beginning from the transition area between the convexity and the edge up unto the peripheral edge area. This is intended without the size of the aperture angle changing as a result of an adaptation to the height of the edge of the upper and lower sliding partner. This “dovetail” shape of the edge of the middle sliding partner increases the safeguard against dislocation.

As per invention, a shape for the upper and lower sliding partner is intended for three part-prosthesis, in which the peripheral edge areas are complete or partly hook-shaped, perpendicular, otherwise angular, curved or a combination thereof in direction of the other outer sliding partner. In this design, the edge of the middle sliding partner is narrower there, so that the middle sliding device is partly or completed covered by the feature of one or both outer sliding partners, in order to prevent a slip-out of the middle sliding device. Advantageously the edge of the middle sliding partner is adapted in such a way to the shape of the edge of an outer sliding partner, that during terminal gap-closure as high as possible an area of the articulating sliding partners comes into contact.

Further it is intended for an intervertebral disc prosthesis, as per invention, that the outer circumferences of the upper and lower sliding partner may taper off from dorsal to ventral (lumbar spine) or from ventral to dorsal (cervical spine) in a transversal view. This tapering off of the outer circumferences of the upper and lower sliding partner may laterally be in the form of identical curves and is preferably a segment of a circle. Where necessary, area and shape of the outer circumference of the upper and lower sliding partner can be equal or unequal and thus adapted by this to the size of the respective vertebral body to which they are assembled.

The tapering off shape of the circumference of the upper and lower sliding partner is constructed in the shape of identical curves and corresponds on the whole to the for the prosthetic plates applicable area of a vertebral body in a transversal view and leads in that way to an optimal use of the area being at disposal for anchoring the sliding partners with the aim of using a maximized area for load transfer acting on the sliding partners.

Adaptations to the sliding partners, as per invention, of the intervertebral disc prosthesis are further intended, in which upper and/or lower sliding partner are built in such a way in a frontal and/or sagittal section, that the out- and inside of the upper and/or lower sliding partner are parallel or not parallel to each other. By this measure, as per invention, an intervertebral disc prosthesis, as per invention, can be adapted to vertebral body endplates, which are not standing parallel in a frontal view or which, in a sagittal view, should build an optimal lordosis and positioning of the sliding areas. The adaptation to existing asymmetries is not only achieved with the angled sliding core alone, but rather with the upper and lower sliding partner as well, particularly in intervertebral spaces with strong asymmetries. It is therefore further feasible, that the sliding core balances an asymmetry in one direction and the plates correct an asymmetry in another direction.

For a reliable anchorage of the implants within the intervertebral space, a marginal and/or plane interdigitation of the outer sides of the upper and lower sliding partner serves for the connection with an upper or lower vertebral body. The outer sides themselves are flat or convex in shape and it is possible to coat the interdigitation or the vertebra-directed surfaces with or without interdigitation bio-actively or blunt. To minimize the risk of fracturing the vertebral body, a fixation with three ventrally arranged and two dorsally placed anchoring teeth is preferred. As an alternative, laterally continuously arranged rows of teeth are favoured for an improved guidance of the upper and lower sliding partner during implantation between the vertebral bodies, because the forceps of the surgeon can grip in the middle gap between the rows of teeth or into holes of the upper and lower sliding partner at the level with the teeth.

To facilitate implantation or explantation of the intervertebral disc prosthesis, the upper and lower sliding partner is furbished with a provision for instruments in a further design. These provisions preferably comprise holes or moulds, into which the required instrument of the surgeon can grip so that a secure fixation of the respective sliding partner is possible.

Furthermore, as absolute measurements for an intervertebral disc prosthesis, as per invention, a maximal breadth (frontal view) of 14 to 48 mm, including about 16 mm, about 18 mm, about 20 mm, about 22 mm, about 24 mm, about 26 mm, about 28 mm, about 30 mm, about 32 mm, about 34 mm, about 36 mm, about 38 mm, about 40 mm, about 42 mm, about 44 mm or about 46 mm, a maximal depth (sagittal view) of 11 to 35 mm, including about 13, about 15 mm, about 17 mm, about 19 mm, about 21 mm, about 23 mm, about 25 mm, about 27 mm, about 29 mm, about 31 mm, about 33 mm, and a maximal height of 4 to 18 mm, including about 6 mm, about 8 mm, about 10 mm, about 12 mm, about 14 mm or about 16 mm, are intended. These measurements are taken from the natural conditions of the lumbar and cervical spine and assure that the situation with an intervertebral disc prosthesis, as per invention, comes very close to the in vivo situation.

Further, for an intervertebral disc prosthesis, as per invention, one or more X-ray contrast giving markers are provided, which are located under the surface of each of the non X-ray contrast giving parts of the prosthesis. That way it is possible to exactly control the position of these parts of the intervertebral disc prosthesis after the implantation. Furthermore it is possible to check, if these parts have changed their position or if they are still in the right position in defined timely intervals.

Further useful measures are described in the dependent claims; the invention is described in the following by design-examples and the ff. figures.

### BRIEF DESCRIPTION OF THE DRAWINGS

FIG. 1 schematic transverse section of a sliding partner with concavity

FIG. 2 a-c schematic view of a median frontal section of a two-part prosthesis, as per invention, with angled sliding core 13 and upper and lower sliding partners 11, 12:
a: upper sliding partner not inclined
b: gap-closure to the left of both sliding partners
c: gap-closure to the right of both sliding partners

FIG. 3 a-c schematic view of a median sagittal section of a two part intervertebral disc prosthesis with angled sliding core:
a: upper sliding partner without inclination
b: gap-closure to the left of both sliding partners
c: gap-closure to the right of both sliding partners

FIG. 4 a-c schematic view of a median frontal section of a three part disc prosthesis, as per invention, with angled sliding core:
a: sliding partners without inclination
b: gap-closure to the left of both sliding partners
c: gap closure to the right of both partners

FIG. 5 a-c schematic view of a median sagittal section of a three part intervertebral disc prosthesis, as per invention, with angled sliding core:
a: sliding partners without inclination
b: gap-closure to the left of both sliding partners
c: gap closure to the right of both partners

FIG. 6 a-c schematic depiction of different shapes of the upper and lower sliding partners for the lumbar spine

FIG. 7 a, b schematic depictions of the arrangement of the anchoring teeth on the outsides of the upper and lower sliding partner for the lumbar spine

FIG. 8 a-g schematic spatial depiction of different parts of as well as an assembled intervertebral disc prosthesis, as per invention.

### DESCRIPTION OF VARIOUS AND PREFERRED EMBODIMENTS

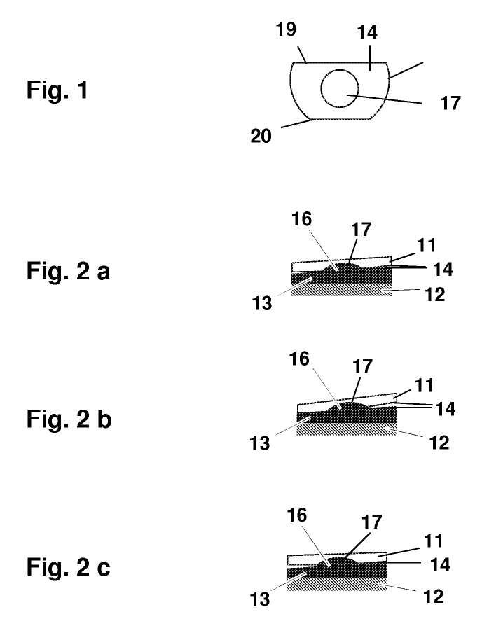

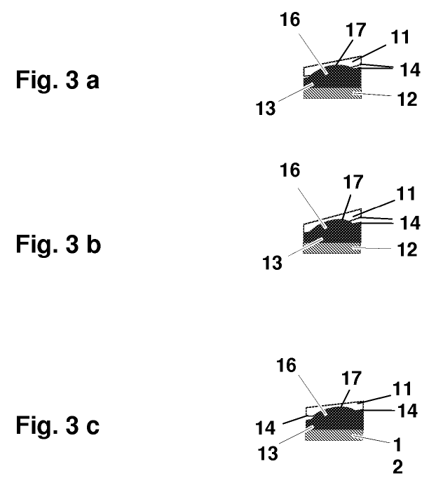

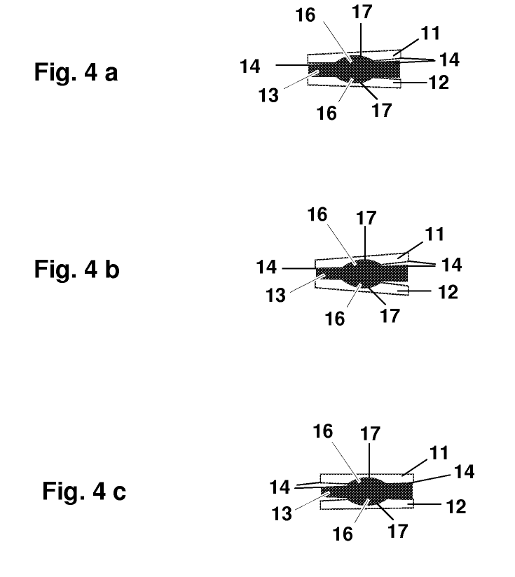

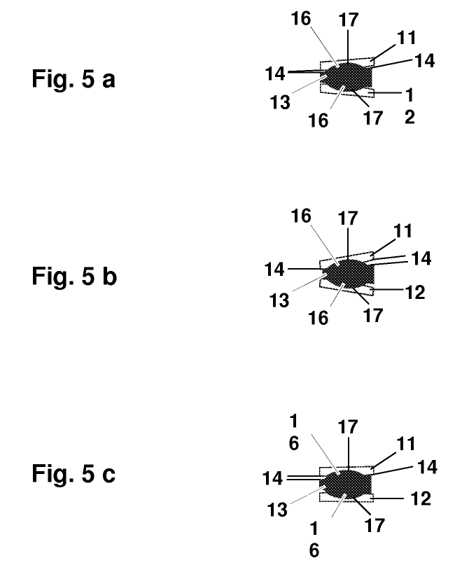

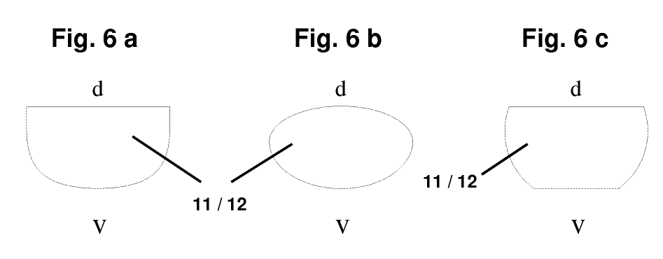

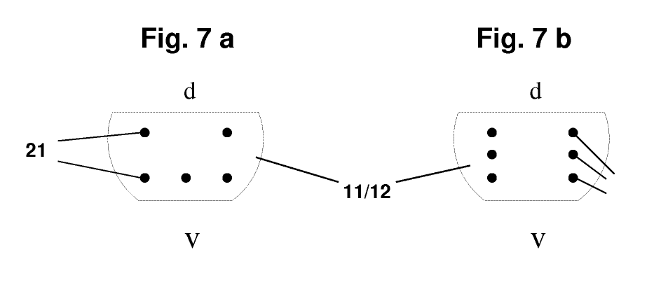

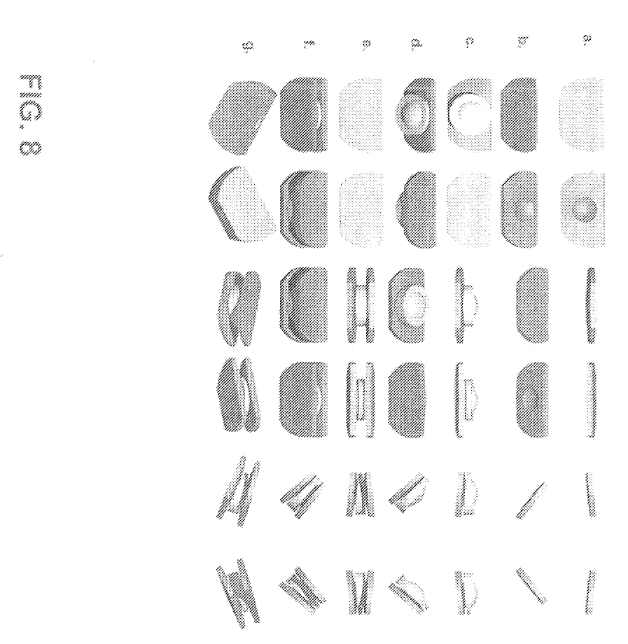

FIG. 1 shows a view of the inside of a sliding partner 11, 12 with a concavity 17, which is surrounded by a edge 14. The shape of the concavity 17 corresponds to the recess of a sphere. A sliding partner 11, 12, whose outer shape tapers off from the dorsal side 19, to the ventral side 20 is intended for the lumbar spine. For the cervical spine the outer shape tapers off from ventral to dorsal. In the schematic view, only dorsal and ventral sides need to be exchanged. In the depicted design, the tapering off takes place circularly; other shapes are feasible. FIG. 6 a-c show further designs of the outer shape of the upper and lower sliding partner 11, 12.

FIGS. 2 a-c show a schematic view of a median section of a two part intervertebral disc prosthesis, as per invention, with an angled sliding core 13 and upper and lower sliding partner 11, 12. Lower sliding partner 12 and angled sliding core 13 can be constructed in one piece, permanently or permanently but reversibly assembled. In FIGS. 2 a-c the laterolateral inclination of the edge 14 as well as of the convexity 16 of the angled sliding core 13 can be seen. The convexity 16 articulates with the concavity 17 of the upper sliding partner 11. The total area, comprising the edge 14 and the convexity 16 of the sliding core 13 is inclined, with respect to a horizontal and has a defined angle. The surface of the edges 14 on both side of the convexity 16 lie in the same straight line. The convexity and the corresponding concavity can, with respect to the inclination, be symmetrical or asymmetrical.

FIG. 2 a shows the inclined outside of the upper sliding partner 11, which is not the result of an inclination of the upper sliding partner 11 to one side of the edge 14 of the asymmetric sliding core 13, but rather of the laterolateral inclination of the angled sliding core 13. The gaps between the edge 14 of the angled sliding core 13 and the edge 14 of the upper sliding partner 11 are of the same size to both sides of the convexity 16 and concavity 17.

FIG. 2 b shows a gap-closure between the edges 14 on the left side of the convexity 16 and concavity 17 of the upper and lower sliding partner 11, 12 and 13, whereas FIG. 2 c shows a one-sided gap-closure on the right side of convexity 16 and concavity 17.

FIGS. 3 a-c each show a median sagittal section of a two part intervertebral disc prosthesis, as per invention, with angled sliding core 13 and upper and lower sliding partner 11, 12. The edges 14 and the convexity 16 of the angled sliding core 13 in these three figures have an inclination from dorsal to ventral or from ventral to dorsal with respect to a horizontal. This inclination of the angled sliding core 13 is the reason for the inclination of the outside of the upper sliding partner 11, which articulates via the concavity 17 with the angled sliding core 13 without the upper sliding partner 11 being inclined towards the edge 14 of the asymmetric sliding core 13. Such an inclination of the upper sliding partner to the dorsal or ventral edge 14 of the angled sliding core 13 with a gap-closure is depicted in FIGS. 3 b and c. Depending on the design of the intervertebral disc prosthesis, as per invention, and on tolerances, gap-closures may however also be incomplete.

FIGS. 4 a-c show a schematic view of a median frontal section of a three part intervertebral disc prosthesis, as per invention, with angled sliding core 13 and upper and lower sliding partner 11, 12. The angled sliding core 13 has a sliding surface on an upper and a lower side with a convexity 16 and a edge 14. With respect to a horizontal, both edges 14 are inclined. The edges may or may not each lie on a joint straight line. On the whole, a wedge shape is to be seen on inspection of the edge 14 from upper and lower sliding surface of the sliding core 13. The convexities 16 of the upper and lower sliding surface each articulate with the concavity 17 of the upper or lower sliding partner 11, 12. The convexities and the corresponding concavities may in conjunction with the inclination be symmetrical or asymmetrical. The total area of the sliding surface, comprising edge 14 and convexity 16 of the asymmetrical sliding core 13 is inclined laterolaterally with respect to a horizontal and has a defined angle. With respect to the horizontal this angle may be of the same or different size above and below.

The shape of the convexity 16 corresponds to the cap of a sphere and that of the articulating concavity 17 of an upper and lower sliding partner 11, 12, the inside of a sphere, as is depicted in FIG. 1.

FIG. 4 a shows a three part prosthesis, in which the upper and lower sliding partner 11,12 are not inclined to one side of the angled sliding core 13. On both sides of the convexity 16 and concavity 17 a gap with an identical aperture angle is visible in the upper as well as lower articulation surface. In FIG. 4 b, the upper and lower sliding partners 11, 12 are each inclined towards the left edge 14 of the angled sliding core 13, which in the illustration of the design leads to a gap-closure to the left of the convexities 16 and concavities 17. FIG. 4 c shows a gap-closure of the edges 14 to the right of the convexities 16 and concavities 17.

FIGS. 5 a-c each show a median sagittal section of a three part intervertebral disc prosthesis, as per invention, with angled sliding core 13 and upper and lower sliding partner 11, 12. The edges 14 and the convexities 16 of the angled sliding core 13 have an inclination of the upper and lower sides from dorsal to ventral or from ventral to dorsal with respect to a horizontal in all three figures. This inclination of the angled sliding core 13 is the reason for the inclination of the outsides of upper and lower sliding partner 11, 12, which each articulate via the concavities 17 with the convexities 16 of the sliding core 13, without upper and/or lower sliding partner 11, 12 being inclined towards the edge 14 of the sliding core 13. Such an inclination of the upper sliding partner towards the dorsal or ventral edge 14 of the angled sliding core 13 is depicted in FIGS. 5 b and c.

FIGS. 6 a-c each show a top view onto schematic alternative designs of the circumference of upper and lower sliding partner 11, 12. The small letters indicate the orientation with respect to the dorsoventral orientation of the plates for the lumbar spine (d=dorsal; v=ventral), which is however reversed for the cervical spine (v=dorsal; d=ventral).

FIGS. 7 a and 7 b show alternative arrangements of the anchoring teeth 21 on the outside of the upper and lower sliding partner 11, 12. Again the orientation of the sliding partners with respect to the dorsoventral orientation is indicated by the small letters (d=dorsal; v=ventral). Dorsally in the middle no anchoring teeth 21 are intended, because this results on one hand in protecting the vertebral bodies and on the other hand facilitates the implantation. For the cervical spine the reversed orientation is also without middle dorsal anchoring teeth 21.

FIGS. 8 a to 8 g show different perspectives of one embodiment of an upper and/or lower sliding partner 11, 12 (FIGS. 8 a and b), an upper and/or lower sliding partner 11, 12 assembeled with an embodiment of a sliding core 13 having slanted edges (FIGS. 8 c and d), and an assembled intervertebral disc prosthesis comprising an upper and lower sliding partner 11, 12 and having a sliding core 13 positioned in between.

The shown designs of a two-part as well as a three-part intervertebral disc prosthesis, as per invention, in the figures are only exemplary and not definite. Once given the above disclosure, many other features, modifications, and improvements will become apparent to the skilled artisan. Such other features, modifications, and improvements are therefore considered to be part of this invention. The angled sliding core 13 is also object of the invention and therefore does not only stand in conjunction with a two- or three part intervertebral disc prosthesis. The convexity or concavity of a sliding core 13, as per invention, can be selected or dimensioned in such a way that it is compatible with other prostheses. This makes it possible to exchange the angled sliding core in primary or revision surgery with the sliding core of an existing prosthesis. The necessity to remove well ingrown sliding partners, which are well assembled to the vertebral bone, is also not given.

### REFERENCE NUMBERS

11 upper sliding partner
12 lower sliding partner
13 angled sliding core or sliding partner
14 edge
16 convexity
17 concavity
19 dorsal side of the sliding partner
20 ventral side of sliding partner
21 anchoring teeth

1. Sliding core positioned in between the inner sides of an upper and a lower sliding partner of a two- and three part functional intervertebral disc prosthesis for correcting angular positions-between vertebral endplates, for maintaining or improving of function of a motion segment of the lumbar and cervical spine, wherein, depending on the design of a convexity and/or concavity on the upper and/lower side, one or two articulating sliding surface(s) between sliding core and inside(s) of the upper and/or lower sliding partner are formed, and the sliding core is asymmetrically angled in a way that, in at least one vertical section, at least one sliding surface of the sliding core is inclined at a defined angle (angle of inclination) towards a fictitious horizontal, and in the case of two articulating sliding surfaces, the angles of inclination above and below the fictitious horizontal are equal or different and the upper and lower convexity are symmetric or asymmetric.

2. Sliding core according to claim 1, wherein the angle of inclination of the sliding surfaces towards each other range between 2 and 35 degrees.

3. Sliding core according to claim 1, wherein the convexity and/or concavity spans across the whole upper- and/or lower side of the sliding core.

4. Sliding core according to claim 1, wherein the convexity and/or the concavity each are surrounded by an edge, whose breadth and height are equal or different.

5. Sliding core, according to claim 1, wherein, for a one-sided design of a sliding surface, an opposite side
a. has means for a permanent or permanent but reversible assembly with the upper or lower sliding partner or with a further asymmetrically angled sliding core or a symmetrical sliding core with a one-sidedly designed sliding surface, or
b. permanently or permanently but reversibly assembles to the upper or lower sliding partner or with a further asymmetrically angled sliding core or a symmetrical sliding core with a one-sidedly designed sliding surface.

6. Sliding core, according to claim 1, wherein the sliding core is suited for a permanent, or permanent but reversible connection with an upper or lower sliding partner of an intervertebral disc prosthesis via a tongue and groove assembly, a guiding track and corresponding recess, a snap mechanism, gluing or screwing.

7. Sliding core, according to claim 4, with the sliding core and the edge, or the means for an assembly with a sliding partner are made of the same or different materials.

8. Sliding core, according to claim 1 comprising the same or different materials as the articulating sliding partners with which it forms a three part intervertebral disc prosthesis.

9. Sliding core, according to claim 1, wherein the surface of the sliding core is equally or differently coated as the articulating sliding partners with which it forms a three part intervertebral disc prosthesis.

10. Sliding core, according to claim 4, wherein, as a safeguard against a slip out from a intervertebral disc prosthesis during terminal gap-closure of the sliding partners, a stop is positioned on the outside, which is, on at least the upper or lower side higher than the edge of the sliding core.

11. Sliding core according to claim 4, wherein the sliding core has a stop on its upper and/or lower side as a safeguard against a slip-out from the intervertebral disc prosthesis during terminal gap-closure of the sliding partners, which, on the upper- and/ or lower side, is higher than the edge of the sliding core and which is guided within a slot of the edge region of the upper and/or lower sliding partner with a clearance necessary for a maximal sliding motion of the sliding partners.

12. Sliding core according to claim 4, wherein, as an additional safeguard against a slip out from the prosthesis during a gap-closure of all three sliding partners, the edge of the sliding core partly or totally increases in height from the transition area of the convexity to the periphery.

13. Sliding core according to claim 1, wherein one or both sliding surfaces are made of plane, spherical, cylindrical, ellipsoid, spindle-shaped or oval surfaces or a combination thereof suitable for a sliding motion and wherein, for a sliding core with sliding surfaces on upper and lower side, the respective sliding surfaces are designed identically or differently with respect to the shape and/or direction of the enabled sliding motion.

14. Sliding core according to claim 1, wherein the sliding core has one or more radiolucent tags beneath its surface.

15. Intervertebral disc prosthesis for the compensation of angular positions between vertebral endplates, for maintaining or improving a function of a motion segment of the lumbar and cervical spine, comprising
an upper sliding partner with an upper exterior side permanently assembled to an upper vertebral body,
a lower sliding partner with a lower exterior side, said exterior side being permanently assembled to a lower vertebral body, and
a sliding core positioned between the inner sides of the upper and lower sliding partner, wherein
a. depending on the design of a convexity and/or concavity on the upper and/or lower side of the sliding core, one or two articulating surfaces are formed between sliding core and inner side(s) of the upper and/or lower sliding partner, and
b. the sliding core is designed in such an asymmetrically angled way, that in at least one vertical section at least one sliding surface of the sliding core is inclined at a defined angle (angle of inclination, aperture angle) towards a fictitious horizontal, and
c. in the case two articulation surfaces are present, the angles of inclination above and below the fictitious horizontal are equal or different and upper and the lower convexity are designed symmetrically or asymmetrically.

16. Intervertebral disc prosthesis according to claim 15, wherein the angle of inclination of the sliding surfaces towards each other ranges between 2 degrees and 35 degrees.

17. Intervertebral disc prosthesis, according to claim 15, wherein the convexity(ies) and/or concavity(ies) span across the complete upper and/or lower side of the sliding core.

18. Intervertebral disc prosthesis, according to claim 15, wherein the convexity(ies) and/or concavity(ies) are each surrounded by an edge with equal or different breadth and height.

19. Intervertebral disc prosthesis, according to claim 15, wherein the asymmetrically angled sliding core and the sliding partners are constructed in one piece.

20. Intervertebral disc prosthesis, according to claim 15, wherein the sliding partners and/or the asymmetrically angled sliding core each comprise two permanently, or permanently but reversibly assembled parts, or wherein the asymmetrically angled sliding core is permanently or permanently but reversibly assembled to a sliding partner and side opposing the convexity or concavity has means for a permanent or permanent but reversible assembly.

21. Intervertebral disc prosthesis, according to claim 15, wherein the sliding partners and/or the sliding core as well as the parts connected to them are made of the same or different materials.

22. Intervertebral disc prosthesis, according to claim 15, wherein the surfaces of the sliding partners and/or the sliding core are equally or differently coated.

23. Intervertebral disc prosthesis, according to claim 20, wherein a groove/spring-assembly, a guide rail and corresponding recess, a snap mechanism, gluing or screwing provides for the permanent or permanent but reversible assembly.

24. Intervertebral disc prosthesis, according to claim 15, wherein one or both sliding surfaces of the asymmetrically angled sliding core are made of plane, spherical, cylindrical, ellipsoid, spindle-shaped or oval surfaces or a combination thereof, which allow a sliding motion with an articulating partner and wherein, for a sliding core with sliding surfaces on the upper and lower surface, the sliding surfaces are designed identically or differently with respect to the shape and/or direction of an enabled sliding motion.

25. Intervertebral disc prosthesis, according to claim 18, wherein the maximal possible angle of inclination (aperture angle) between the upper and lower sliding partner including the sliding core depends on
a. design of the convexity(ies) and corresponding concavity(ies) with respect to geometry of the sliding surfaces, height and radius of curvature,
b. shape and extent of the angled asymmetry of the sliding core, and
c. the design of the edge.

26. Intervertebral disc prosthesis, according to claim 15, wherein a maximal aperture angle during one-sided gap-closure of the sliding partners in extension or flexion lies between 6 degrees and 10 degrees and during one-sided lateral gap-closure between 3 degrees and 6 degrees with an additional tolerance of 3 degrees in every direction.

27. Intervertebral disc prosthesis according to claim 15, wherein the convexity(ies) and the respective corresponding concavity(ies) are dorsally displaced up to 4 mm away from the median sagittal section.

28. Intervertebral disc prosthesis according to claim 18, wherein the edges of the sliding partners end outwardly perpendicular, otherwise angled, curved or a combination of straight, curved, and/or angular.

29. Intervertebral disc prosthesis, according to claim 18, wherein, as an additional safeguard for the sliding core with edge against a slip-out out of the prosthesis during a gap closure of the sliding partners, a stop is part of the edge of the sliding core, which is located outside the upper and/or lower sliding partner, wherein the stop on at least an upper or lower side is higher than the edge of the sliding core.

30. Intervertebral disc prosthesis according to claim 18, wherein, as an additional safeguard for the sliding core with edge against a slip-out out of the prosthesis during a gap-closure of the sliding partners, a stop is part of the edge of the sliding core, wherein the stop is higher on its upper and/or lower side than the edge of the sliding core and is guided within a groove in the edge area of the upper and/or lower sliding partner with clearance necessary for the maximal sliding motion of the sliding partners.

31. Intervertebral disc prosthesis according to claim 18, wherein, as an additional safeguard for the sliding core with edge against a slip-out out of the prosthesis during a gap closure of all three sliding partners, the edge of the sliding core increases partly or totally in height from a transition area of the convexity to the periphery, wherein the edge of the upper and/or lower sliding partner levels off by the same amount.

32. Intervertebral disc prosthesis according to claim 15 or 18, wherein, as an additional safeguard for the sliding core against a slip-out out of the prosthesis during a gap closure of the three sliding partners, the most outward edges of the upper and/or lower sliding partner are completely or partially hook-shaped, perpendicular, otherwise angular, curved or a combination thereof in the direction of the other lower and/or upper sliding partner.

33. Intervertebral disc prosthesis according to claim 15, wherein surface and shape of an outer circumference of the upper and lower sliding partner are equal or unequal and can thereby be adapted to the corresponding size of the vertebral body to which they are to be assembled.

34. Intervertebral disc prosthesis according to claim 15, wherein the upper and/or lower sliding partner are designed so that in a frontal and/or sagittal view an outside and inside of the upper and/or lower sliding partner are parallel or non-parallel.

35. Intervertebral disc prosthesis according to claim 15, wherein the upper and lower sliding partner are plane or convex and coated bio-actively or blunt on the outer surface and have for their primary anchorage with the vertebral bodies anchoring teeth arranged in rows, that are either arranged from dorsal to ventral laterally straight or at an incline or dorsal and ventral in lateral alignment, wherein in a respective dorsal row the anchoring teeth are arranged only laterally.

36. Intervertebral disc prosthesis according to claim 15, wherein the upper and/or lower sliding partner have means for an instrument to grip them for implantation or explantation.

37. Intervertebral disc prosthesis according to claim 15, wherein the prothesis has a maximal breadth (frontal view) of 14 to 48 mm, a maximal depth (sagittal view) of 11 to 35 mm and a maximal height of 4 to 18 mm.

38. Intervertebral disc prosthesis according to claim 15, suitable for implantation into a lumbar spine, and wherein an outer circumference of the upper and lower sliding partners tapers off ventrally in transversal view.

39. Intervertebral disc prosthesis according to claim 15, suitable for implantation into a cervical spine, and wherein an outer circumference of the upper and lower sliding partners tapers off dorsally in transversal view.

40. Intervertebral disc prosthesis according to claim 38, wherein the tapering off of the outer circumference of the upper and lower sliding partner, has laterally identical curvation or is asymmetric.

41. Intervertebral disc prosthesis according to claim 15, wherein the non-X-ray contrast giving parts of the prosthesis are each marked under their surface with one or more radiolucent tags.

42. Intervertebral disc prosthesis according to claim 39, wherein the tapering off of the outer circumference of the upper and lower sliding partner, has laterally identical curvation or is asymmetric.
